(19) United States
(12) Patent Application Publication
(10) Pub. No.: US 2007/0166358 A1
(43) Pub. Date: Jul. 19, 2007

(54) Nasal mucus absorber pad

(76) Inventor: Hyonho Chong, Northridge, CA (US)

Correspondence Address: PARK LAW FIRM, 3255 WILSHIRE BLVD, SUITE 1110, LOS ANGELES, CA, 90010, US

(21) Appl. No.: 11/333,687

(22) Filed: Jan. 17, 2006

(51) Int. Cl. A61K 9/70; B32B 27/04

(52) U.S. Cl. 424/443; 442/123

(57) ABSTRACT

A nasal mucus absorber pad for a person includes a fluid absorber, a backing part, and a fixing part. The fluid absorber, for absorbing bodily fluids, includes an inner side, an outer side, a top edge, a bottom edge, and side edges. The backing part, for backing the outer side of the fluid absorber, includes a first surface, a second surface, a center portion, and side portions, wherein the first surface is attached to the outer side of the fluid absorber. The fixing part connected to the backing part, for fixing the nasal mucus absorber pad to the skin of the person, includes sticky portions. The fluid absorber is adapted to absorb and retain the bodily fluids stopping further flow, and the surface contour of the inner side of the fluid absorber is adapted for wearing the nasal mucus absorber pad across the philtrum of the person under the nose.

### BACKGROUND OF THE INVENTION

The present invention relates to a nasal mucus absorber pad.

More particularly, this invention relates to a nasal mucus absorber pad that absorbs and retains the nasal mucus to prevent it from flowing or dripping.

Still more specifically, the present invention relates to a nasal mucus absorber pad, which helps to prevent skin irritation caused by rubbing and wiping of nose.

Still more specifically, the present invention relates to a nasal mucus absorber pad that is portable and disposable and helps the user to go to sleep, work, and perform other daily routines without being bothered by the nasal mucus.

Nasal mucus is so common on people suffering from a regular cold or some certain sorts of allergy. In such cases, nasal bodily fluid, mucus, forms inside the nose so that the nose is blocked with it. The nasal mucus then flows/slides down through the nasal passage and the nostrils.

Flowing nasal mucus is much more bothersome than one expects. It keeps the patient from concentrating on anything other than wiping the fluid or blowing the nose. In serious cases, there comes a lot of side effects. Blowing nose may cause a severe headache, sore skin problem around the nostrils, and many other serious health problems.

Even though there are varying mucus removal methods, no device is currently available that extracts the nasal mucus in a satisfying and efficient manner.

There are no prior arts available to solve the problem of handling the nasal mucus in more sanitary, systematic, and practical manners than the present invention does.

The novel Nasal Mucus Absorber Pad, with its adhesive members, is physically applied in close engagement with both nostrils. This is to effectively collect and retain the nasal mucus. With the Nasal Mucus Absorber Pad on, the users can do better with the physical and mental functions in their daily activities more comfortably. Most of all, they can sleep all night with the pad on.

Accordingly, a need for a nasal mucus absorber pad has been present for a long time considering the extent of the sufferings from the perennial cold and the allergy. This invention is directed to solve these problems and satisfy the long-felt need.

### SUMMARY OF THE INVENTION

The present invention contrives to solve the disadvantages of the prior art.

An object of the invention is to provide a nasal mucus absorber pad.

Another object of the invention is to provide a nasal mucus absorber pad that absorbs and retains the nasal mucus to prevent it from flowing or dripping.

Still another object of the invention is to provide a nasal mucus absorber pad, which helps to prevent skin irritation caused by rubbing and wiping of nose.

Still another object of the invention is to provide a nasal mucus absorber pad that is portable and disposable and helps the user to go to sleep, work, and perform other daily routines without being bothered by the nasal mucus.

A nasal mucus absorber pad for a person includes a fluid absorber, a backing part, and a fixing part.

The fluid absorber, for absorbing bodily fluids, includes an inner side, an outer side, a top edge, a bottom edge, and side edges.

The backing part, for backing the outer side of the fluid absorber, includes a first surface, a second surface, a center portion, and side portions, wherein the first surface is attached to the outer side of the fluid absorber.

The fixing part connected to the backing part, for fixing the nasal mucus absorber pad to the skin of the person, includes sticky portions.

The fluid absorber is adapted to absorb and retain the bodily fluids stopping further flow, and the surface contour of the inner side of the fluid absorber is adapted for wearing the nasal mucus absorber pad across the philtrum of the person under the nose.

The fluid absorber includes silica gel of a predetermined portion of the total mass of the mucus absorber. The predetermined portion is determined by the amount of bodily fluids to absorb.

The predetermined portion is about from ten (10) to fifty (50) percent.

The fluid absorber is tapered, and the top edge is thicker than the bottom edge.

The backing part includes fabric and plastic.

The second surface of the backing part has preferably skin color and skin texture.

The sticky portion of the fixing part is provided on the side edges of the first surface of the backing part.

The fixing part further includes two side bands extending from the side edges, and the sticky portions are provided on the side bands.

The nasal mucus absorber pad is substantially rectangular with a top edge line, a bottom edge line, and side edge lines. top edge line is adapted to accommodate the shape of the nostrils and the philtrum.

The side edges of the fluid absorber and the side portions of the backing part are extended to the side of the nose for absorbing and storing the fluid flowing sideways.

The fixing part further includes a peel-off part for protecting the sticky portions before the usage.

The fluid absorber further includes medicine to help to alleviate the bodily fluid-causing illness of the person. The medicine includes chemical compounds to help the clogged nose and a runny nose. The medicine includes extraction from herbs. The herbs include mint leaf, ginger root, and tangerine peel. The acting components of the medicine is vaporized by the body temperature of the person and breathed into the nostrils.

The fluid absorber includes an absorbing material for absorbing the bodily fluid and a diffusing material for spreading the absorbed bodily fluid substantially evenly across the fluid absorber, and the diffusing material is mixed evenly with the absorbing material. The fluid absorber further includes a cotton material.

The nasal mucus absorber pad may further include a covering pad for covering the fluid absorber, the backing part, and the fixing part in a fashion of mask. And the covering pad is detachably attached to the second surface of the backing part with a hook and loop faster.

The nasal mucus absorber pad may further include a nose covering flip extended from the top edge of the backing part for covering up to the lower part of the nose. The medicine to alleviate the illness may be applied in the nose covering flip.

The nasal mucus absorber pad further includes an absorbing mesh on the top edge of the fluid absorber, which works as an entrance of the nasal mucus into the fluid absorber.

The advantages of the present invention are: (1) the nasal mucus absorber pad is convenient to use due to its compactness and disposability; (2) the nasal mucus absorber pad saves a lot of trouble from the user by absorbing and retaining the problematic and debilitating nasal mucus; and (3) the nasal mucus absorber pad helps to alleviate the symptoms of cold or allergy without any hassles with the busy hands.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a perspective view of a nasal mucus absorber pad according to the present invention;

FIG. 2 is a rear view of the nasal mucus absorber pad with a fixing part;

FIG. 3 is a side view of the nasal mucus absorber pad with its tapered cross-section;

FIG. 4 is a perspective view of a user wearing the nasal mucus absorber pad;

FIG. 5 is a perspective front view of another nasal mucus absorber pad according to the present invention;

FIG. 6 is a perspective rear view of the nasal mucus absorber pad;

FIG. 7 is a perspective front view of the nasal mucus absorber pad with a fixing part attached;

FIG. 8 is a perspective rear view of the nasal mucus absorber pad with a fixing part attached;

FIG. 9 is a side perspective view of a user wearing the nasal mucus absorber pad;

FIG. 10 is a front perspective view of the user wearing the nasal mucus absorber pad;

FIG. 11 is a perspective view of the nasal mucus absorber pad with an extended side portions;

FIG. 12 is a plan view of a covering pad; and

FIG. 13 is a perspective view of the nasal mucus absorber pad with a nose covering flip.

### DETAILED DESCRIPTION OF THE INVENTION

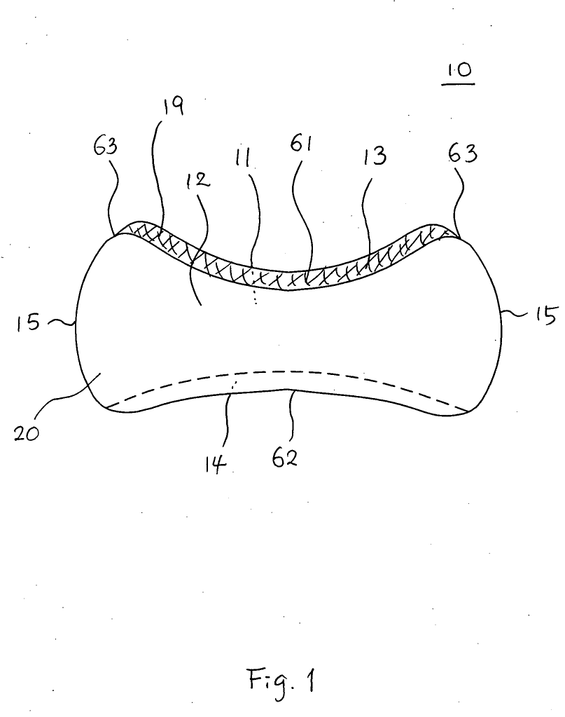

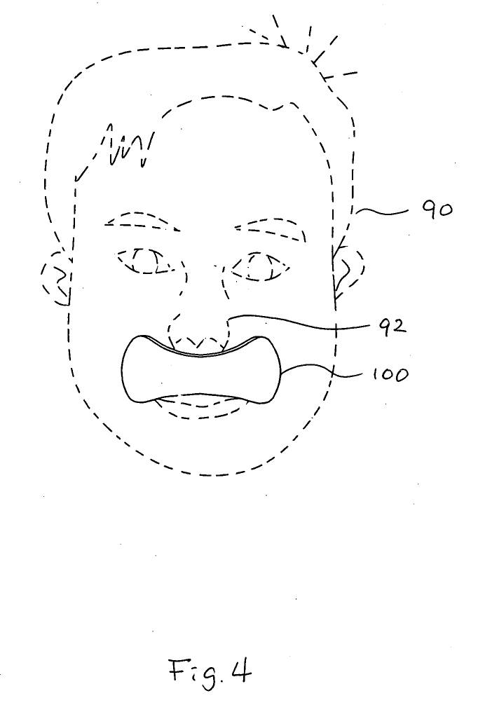

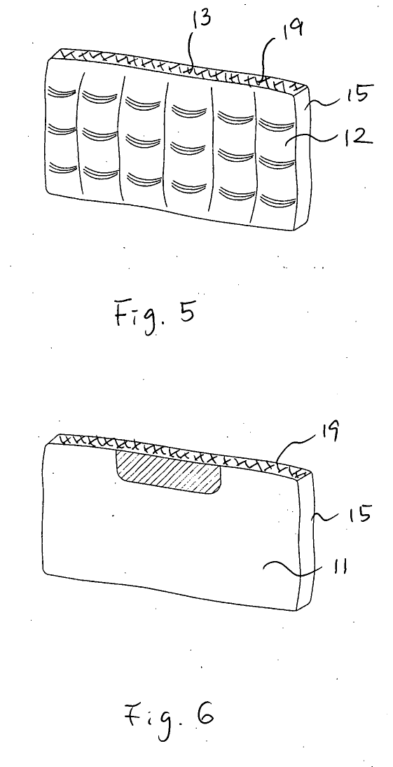

FIGS. 1 through 4 show an embodiment of a nasal mucus absorber pad 100 according to the present invention.

The nasal mucus absorber pad 100 for a person 90 includes a fluid absorber 10, a backing part 20, and a fixing part 30.

The fluid absorber 10, for absorbing bodily fluids, includes an inner side 11, an outer side 12, a top edge 13, a bottom edge 14, and side edges 15.

The backing part 20, for backing the outer side of the fluid absorber 10, includes a first surface 21, a second surface 22, a center portion 23, and side portions 24, wherein the first surface 21 is attached to the outer side 12 of the fluid absorber 10.

The fixing part 30 connected to the backing part 20, for fixing the nasal mucus absorber pad 100 to the skin of the person 90, includes sticky portions 32.

The fluid absorber 10 is adapted to absorb and retain the bodily fluids stopping further flow, and the surface contour of the inner side 11 of the fluid absorber 10 is adapted for wearing the nasal mucus absorber pad 100 across the philtrum 91 of the person 90 under the nose 92.

The fluid absorber 10 includes silica gel of a predetermined portion of the total mass of the mucus absorber 10. The predetermined portion is determined by the amount of bodily fluids to absorb. The predetermined portion is about from ten (10) to fifty (50) percent.

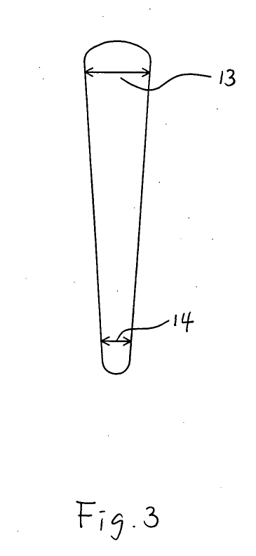

The fluid absorber 10 is tapered, and the top edge 13 is thicker than the bottom edge 14 as shown in FIG. 3.

The backing part 20 includes fabric and plastic.

The second surface 22 of the backing part 20 has preferably skin color and skin texture.

The sticky portion 32 of the fixing part 30 is provided on the side edges 25 of the first surface 21 of the backing part 20.

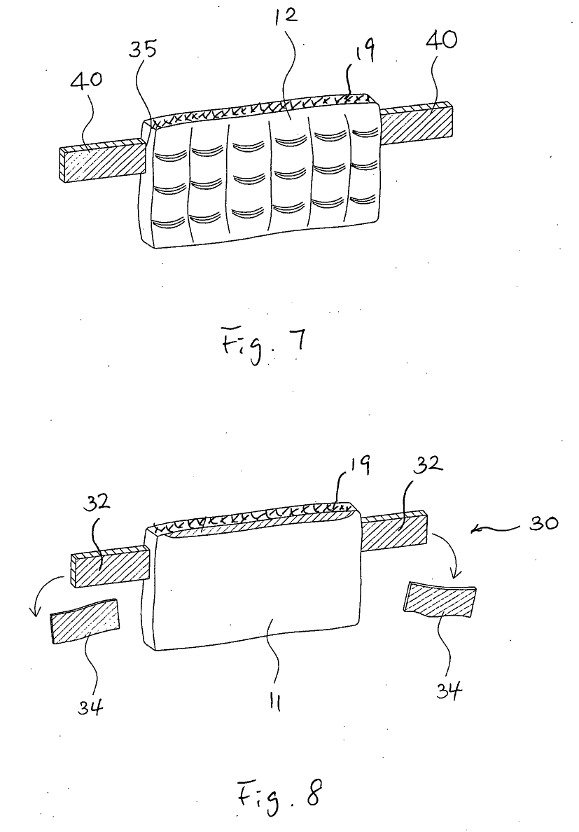

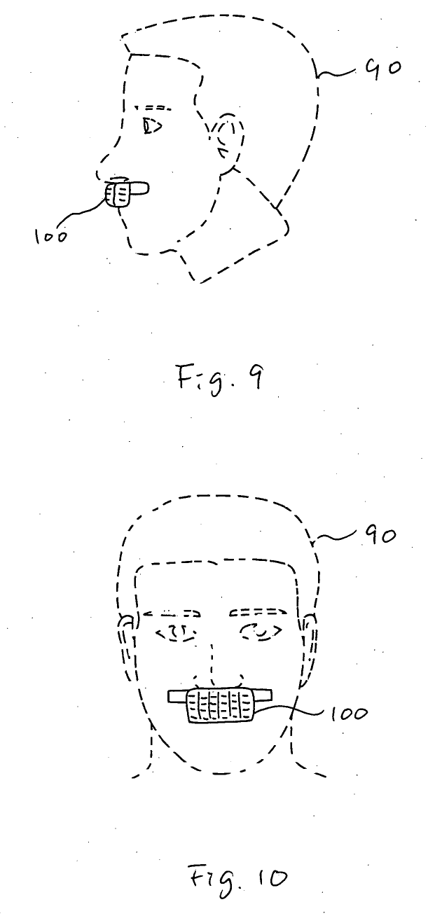

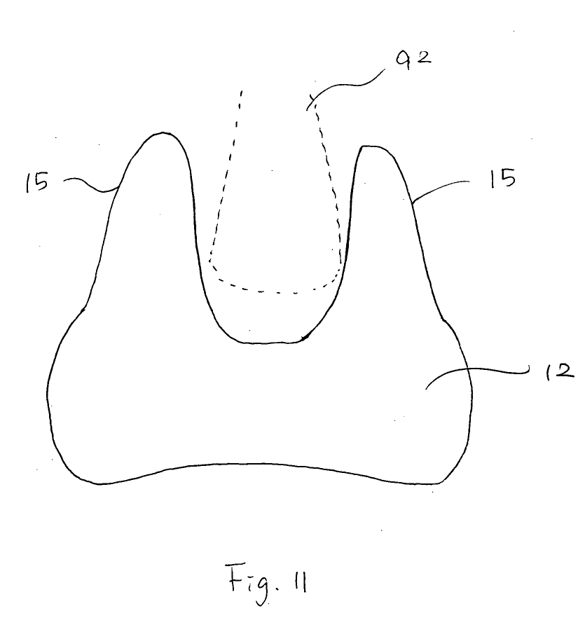

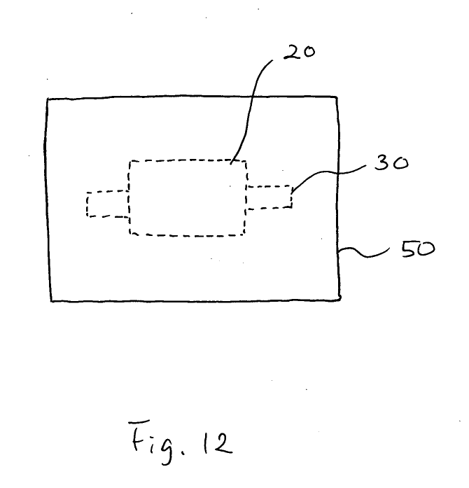

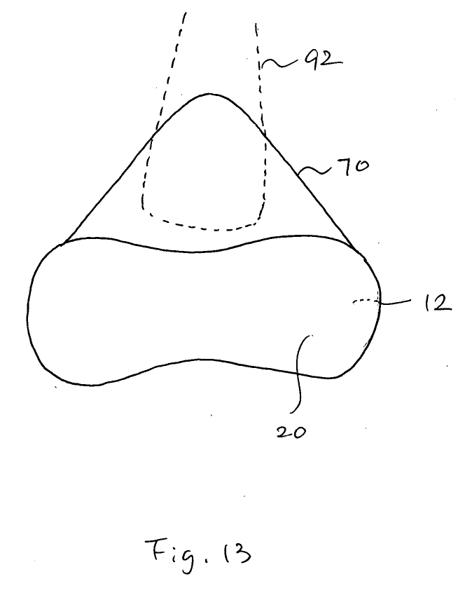

The fixing part 30 further includes two side bands 40 extending from the side edges 35, and the sticky portions 32 are provided on the side bands 40 as shown in FIG. 7 and FIG. 8.

As shown in FIG. 1, the nasal mucus absorber pad 100 is substantially rectangular with a top edge line 61, a bottom edge line 62, and side edge lines 63. The top edge line 61 is adapted to accommodate the shape of the nostrils 93 and the philtrum 91.

The side edges 15 of the fluid absorber 10 and the side portions 24 of the backing part 20 are extended to the side of the nose 92 for absorbing and storing the fluid flowing sideways.

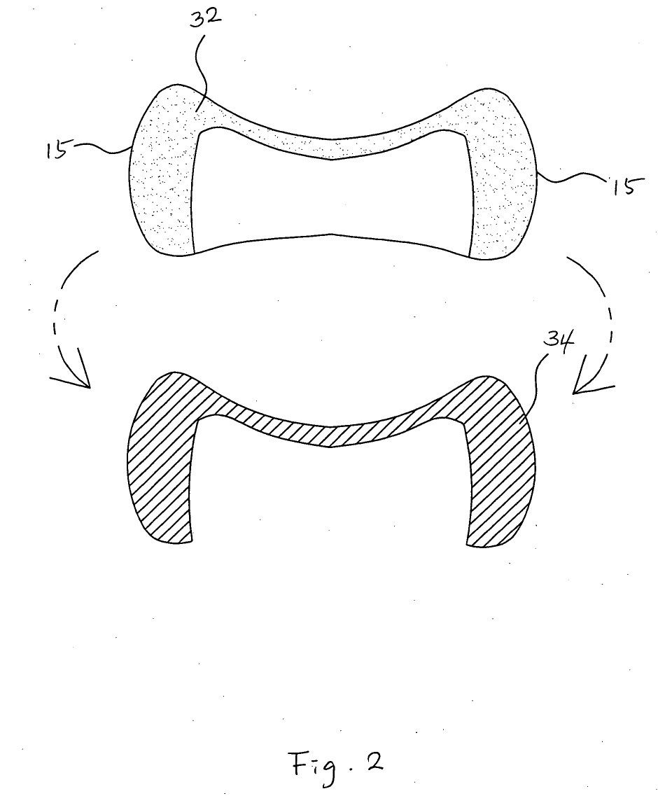

The fixing part 30 further includes a peel-off part 34 for protecting the sticky portions 32 before the usage as shown in FIG. 2 and FIG. 8.

The fluid absorber 10 further includes medicine to help to alleviate the bodily fluid-causing illness of the person. The medicine includes chemical compounds to help the clogged nose and a runny nose. The medicine includes extraction from herbs. The herbs include mint leaf, ginger root, and tangerine peel. The acting components of the medicine is vaporized by the body temperature of the person and breathed into the nostrils.

The fluid absorber 10 includes an absorbing material for absorbing the bodily fluid and a diffusing material for spreading the absorbed bodily fluid substantially evenly across the fluid absorber 10, and the diffusing material is mixed evenly with the absorbing material. The fluid absorber 10 further includes a cotton material.

The nasal mucus absorber pad 100 may further include a covering pad 50 for covering the fluid absorber 10, the backing part 20, and the fixing part 30 in a fashion of mask. And the covering pad 50 is detachably attached to the second surface 22 of the backing part 20 with a hook and loop faster (not shown), preferably Velcro®.

The nasal mucus absorber pad 100 may further include a nose covering flip 70 extended from the top edge of the backing part 20 for covering up to the lower part of the nose 92. The medicine to alleviate the illness may be applied in the nose covering flip 70 as shown in FIG. 13.

The nasal mucus absorber pad 100 further includes an absorbing mesh 19 on the top edge 13 of the fluid absorber 10, which works as an entrance of the nasal mucus into the fluid absorber 10.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A nasal mucus absorber pad for a person comprising:
a) a fluid absorber, for absorbing bodily fluids, having an inner side, an outer side, a top edge, a bottom edge, and side edges;
b) a backing part, for backing the outer side of the fluid absorber, having a first surface, a second surface, a center portion, and side portions, wherein the first surface is attached to the outer side of the fluid absorber; and
c) a fixing part connected to the backing part, for fixing the nasal mucus absorber pad to the skin of the person, having sticky portions,
wherein the fluid absorber is adapted to absorb and retain the bodily fluids stopping further flow, wherein the surface contour of the inner side of the fluid absorber is adapted for wearing the nasal mucus absorber pad across the philtrum of the person under the nose.

2. The nasal mucus absorber pad of claim 1, wherein the fluid absorber comprises silica gel of a predetermined portion of the total mass of the mucus absorber, wherein the predetermined portion is determined by the amount of bodily fluids to absorb.

3. The nasal mucus absorber pad of claim 2, wherein the predetermined portion is about from ten (10) to fifty (50) percent.

4. The nasal mucus absorber pad of claim 1, wherein the fluid absorber is tapered, wherein the top edge is thicker than the bottom edge.

5. The nasal mucus absorber pad of claim 1, wherein the backing part comprises fabric and plastic.

6. The nasal mucus absorber pad of claim 1, wherein the second surface of the backing part has preferably skin color and skin texture.

7. The nasal mucus absorber pad of claim 1, wherein the sticky portion of the fixing part is provided on the side edges of the first surface of the backing part.

8. The nasal mucus absorber pad of claim 1, wherein the fixing part further comprises two side bands extending from the side edges, wherein the sticky portions are provided on the side bands.

9. The nasal mucus absorber pad of claim 1, wherein the nasal mucus absorber pad is substantially rectangular with a top edge line, a bottom edge line, and side edge lines.

10. The nasal mucus absorber pad of claim 9, wherein the top edge line is adapted to accommodate the shape of the nostrils and the philtrum.

11. The nasal mucus absorber pad of claim 1, wherein the side edges of the fluid absorber and the side portions of the backing part are extended to the side of the nose for absorbing and storing the fluid flowing sideways.

12. The nasal mucus absorber pad of claim 1, wherein the fixing part further includes a peel-off part for protecting the sticky portions before the usage.

13. The nasal mucus absorber pad of claim 1, wherein the fluid absorber further comprises medicine to help to alleviate the bodily fluid-causing illness of the person.

14. The nasal mucus absorber pad of claim 13, wherein the medicine comprises chemical compounds to help the clogged nose and a runny nose.

15. The nasal mucus absorber pad of claim 13, wherein the medicine comprises extraction from herbs.

16. The nasal mucus absorber pad of claim 15, wherein the herbs comprise mint leaf, ginger root, and tangerine peel, wherein the acting components of the medicine is vaporized by the body temperature of the person and breathed into the nostrils.

17. The nasal mucus absorber pad of claim 1, wherein the fluid absorber comprises an absorbing material for absorbing the bodily fluid and a diffusing material for spreading the absorbed bodily fluid substantially evenly across the fluid absorber, wherein the diffusing material is mixed evenly with the absorbing material, wherein the fluid absorber further comprises a cotton material.

18. The nasal mucus absorber pad of claim 1, further comprising an absorbing mesh on the top edge of the fluid absorber.

19. The nasal mucus absorber pad of claim 1, further comprising a covering pad for covering the fluid absorber, the backing part, and the fixing part in a fashion of mask, wherein the covering pad is detachably attached to the second surface of the backing part with a hook and loop faster.

20. The nasal mucus absorber pad of claim 1, further comprising a nose covering flip extended from the top edge of the backing part for covering up to the lower part of the nose, wherein the fluid absorber further comprises medicine to help to alleviate the bodily fluid-causing illness of the person, wherein the medicine comprises chemical compounds to help the clogged nose and a runny nose, wherein the medicine comprises extraction from herbs, wherein the medicine is applied in the nose covering flip.
